(19) United States
(12) Patent Application Publication
(10) Pub. No.: US 2023/0241386 A1
(43) Pub. Date: Aug. 3, 2023

(54) IN-THE-EAR (ITE) COIL ALIGNMENT

(71) Applicant: Cochlear Limited, Macquarie University, NSW (AU)

(72) Inventor: Jan Patrick FRIEDING, Grose Vale, NSW (AU)

(21) Appl. No.: 18/010,100

(22) PCT Filed: May 4, 2021
(86) PCT No.: PCT/IB2021/053721

§ 371 Date: Dec. 13, 2022

Related U.S. Application Data
(60) Provisional application No. 63/041,219, filed on Jun. 19, 2020.

(51) Int. Cl. A61N 1/36; A61B 5/00; A61N 1/372; G06T 17/00

(52) U.S. Cl. CPC A61N 1/36039; A61B 5/0062; A61N 1/37229; G06T 17/00; G06T 2210/41

(57) ABSTRACT

Presented herein are techniques for determining an optimal or selected placement for an in-the-ear (ITE) coil configured to be removably positioned within an ear canal of a recipient of an implantable auditory prosthesis that comprises an implantable coil positioned adjacent to the ear canal of the recipient. The optimal placement for the ITE coil is used to fabricate/produce an ITE component that is configured to be worn within the ear canal of the recipient. The ITE component is constructed with an arrangement such that, when the ITE component is fittingly inserted into the ear canal, the ITE coil will be situated at the optimal placement within the ear canal. At the optimal placement, the ITE coil is configured to efficiently communicate with the implantable coil positioned adjacent to the ear canal of the recipient.

### BACKGROUND

### Field of the Invention

The present invention relates generally to techniques for aligning an in-the-ear (ITE) coil with an implantable coil of an implantable medical device.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: obtaining anatomical data associated with an outer ear of a recipient of an implantable auditory prosthesis comprising at least one implantable coil configured to be positioned adjacent to an ear canal of the recipient for operation with an in-the-ear (ITE) coil; obtaining telemetry data indicating an inductive coupling between a test coil assembly inserted into the ear canal of the recipient and the implantable coil; and determining, based at least on the telemetry data, an optimal placement for the ITE coil in the ear canal for operation with the implantable coil.

In another aspect, an in-the-ear (ITE) component for use with an implantable coil configured to be positioned adjacent to an ear canal of the recipient is provided. The ITE component comprises: a body arranged to be inserted in the ear canal of the recipient; and an ITE coil attached to the body, wherein the body has a shape such that, when the body is fittingly inserted into the ear canal, the ITE coil and the implantable coil have a predetermined relative positioning determined based on telemetry data obtained from within the ear canal of the recipient.

In another aspect, a method is provided. The method comprises: obtaining anatomical data associated with an outer ear of a recipient of an implantable auditory prosthesis comprising at least one implantable coil configured to be positioned adjacent to an ear canal of the recipient for operation with an in-the-ear (ITE) coil; inserting a test coil assembly into the ear canal of the recipient; performing a radio-frequency scan of the ear canal with the test coil assembly to generate telemetry data characterizing an inductive coupling between the test coil assembly and the implantable coil at a plurality of different relative positioning there between; and determining, based at least on the telemetry data, a selected position for the ITE coil in the ear canal for operation with the implantable coil.

In another aspect, an auditory prosthesis system is provided. The auditory prosthesis system comprises: an implantable component including a main body configured to be implanted adjacent to an ear canal of a recipient of the auditory prosthesis system, an implantable coil, and a stimulation arrangement electrically connected to the main body; and in-the-ear (ITE) component comprising: an ITE coil configured to be inductively coupled to the implantable coil, and an ear mold arranged to be inserted in the ear canal of the recipient, wherein the ear mold has an arrangement such that, when the ear mold is fittingly inserted into the ear canal, the ITE coil and the implantable coil have a predetermined relative positioning determined based on telemetry data obtained from within the ear canal of the recipient and anatomical data associated with the ear canal of the recipient.

### BRIEF DESCRIPTION OF THE DRAWINGS

Embodiments of the present invention are described herein in conjunction with the accompanying drawings, in which:

FIG. 1A is a schematic diagram of a cochlear implant system, in accordance with certain embodiments presented herein;

FIG. 1B is a functional block diagram of the cochlear implant FIG. 1A, in accordance with certain embodiments presented herein;

FIG. 1C is a schematic cross-sectional view of an in-the-ear (ITE) component of the cochlear implant system of FIG. 1A, in accordance with certain embodiments presented herein;

FIG. 2 is a flowchart illustrating an example method, in accordance with certain embodiments presented herein;

FIG. 3A is front-perspective view of an example ear scanning system for use with certain embodiments presented herein;

FIG. 3B is a rear- perspective view of the example ear scanning system of FIG. 3A;

FIG. 4 is a front-perspective via illustrating an example ear scanning system configured to perform a radio-frequency (RF) scan, in accordance with certain embodiments presented herein;

FIG. 5 is a schematic diagram illustrating rendering of an ear mold, in accordance with certain embodiments presented herein;

FIG. 6 is a schematic cross-sectional view of an ITE component, in accordance with certain embodiments presented herein;

FIG. 7 is a schematic cross-sectional view of another ITE component, in accordance with certain embodiments presented herein;

FIG. 8 is a block diagram of a computing device, in accordance with certain embodiments presented herein;

FIG. 9 is a high-level flowchart illustrating an example method, in accordance with certain embodiments presented herein;

FIG. 10 is a high-level flowchart illustrating another example method, in accordance with certain embodiments presented herein;

### DETAILED DESCRIPTION

Presented herein are techniques for determining an optimal or selected placement for an in-the-ear (ITE) coil configured to be removably positioned within an ear canal of a recipient of an implantable auditory prosthesis that comprises an implantable coil positioned adjacent to the ear canal of the recipient. The optimal placement for the ITE coil is used to fabricate/produce an ITE component that is configured to be worn within the ear canal of the recipient. The ITE component is constructed with an arrangement such that, when the ITE component is “fittingly inserted” into the ear canal, the ITE coil will be situated at the optimal placement within the ear canal. At the optimal placement, the ITE coil is configured to efficiently communicate with the implantable coil positioned adjacent to the ear canal of the recipient.

As used herein, “fittingly inserted” refers to a preferred/intended placement of the ITE component in the ear canal 105 such that the ITE component will remain that that placement (e.g., the ITE components properly fits into the ear canal, given the anatomical characteristics affecting retention, comfort, venting, occlusion management, cosmetics, etc. Stated differently, fittingly inserted is the intended or final position for the ITE component during normal/daily operation.

As used herein, an “optimal” placement for an ITE coil is a placement (position) that provides a selected inductive coupling (selected mutual inductance) between the ITE coil and an implantable coil positioned adjacent to the ear canal of the recipient (e.g., an inductive coupling that is greater than a minimum threshold level). In addition, an “optimal” placement for an ITE coil is a placement that accounts for the anatomical characteristics of the ear canal, including anatomical characteristics affecting retention, comfort, or other recipient-specific considerations. In addition, the “placement” or “position” of an ITE coil can include the lateral/medial location, superior/inferior location, or anterior/posterior location of the ITE coil in the ear canal (e.g., location relative the sagittal, coronal, and or transverse planes), as well as the orientation of the ITE coil (e.g., angular position of the coil relative to sagittal, coronal, and or transverse planes).

Merely for ease of description, the techniques presented herein are primarily described herein with reference to a cochlear implant system. However, it is to be appreciated that the techniques presented herein may also be used with a variety of other implantable medical device systems. For example, the techniques presented herein may be used with other auditory prosthesis systems, including middle ear auditory prosthesis systems (middle ear implant systems), bone conduction device systems, direct acoustic stimulator systems, electro-acoustic prosthesis systems, auditory brain stimulator systems, etc. The techniques presented herein may also be used with systems that comprise or include tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

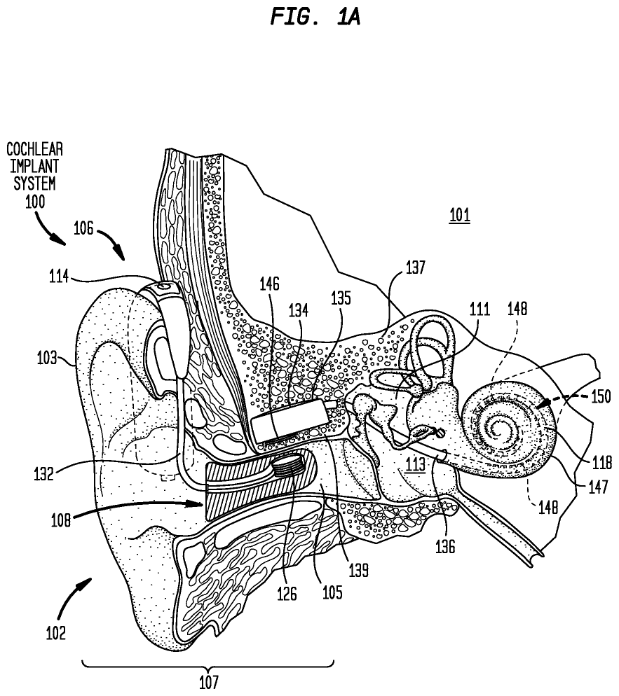

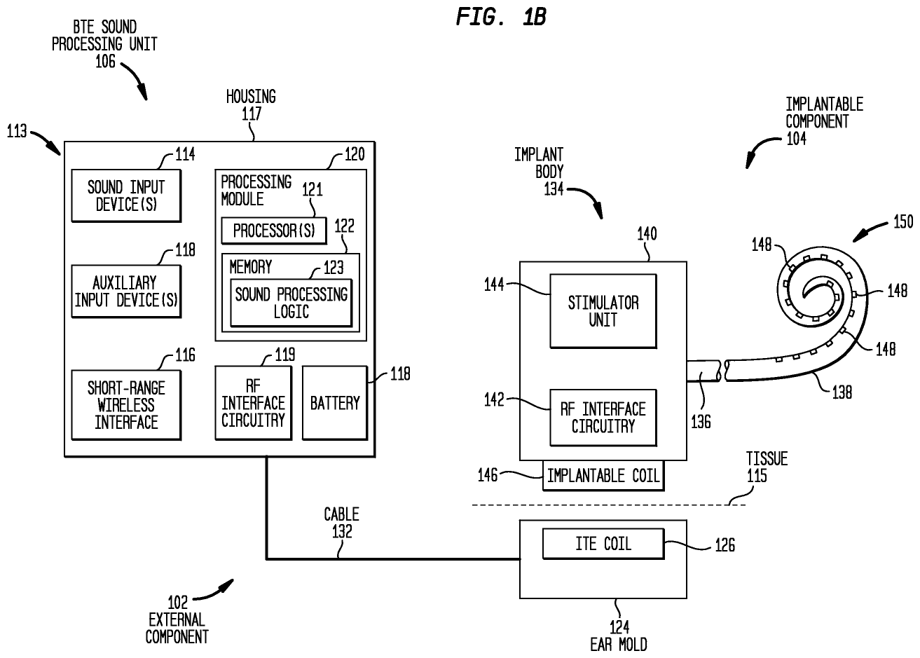

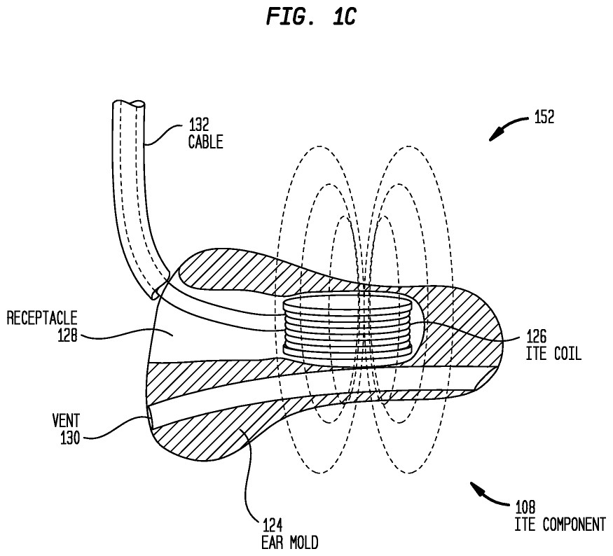

FIG. 1A is a schematic diagram of an example cochlear implant system 100, with which certain embodiments presented herein may be implemented. In FIG. 1A, the cochlear implant system 100 is shown implanted in a recipient 101. FIG. 1B is functional block diagram of the cochlear implant system 100 of FIG. 1A, while FIG. 1C is a partial cross-sectional view of an in-the-ear (ITE) component of the cochlear implant system of FIG. 1A. For ease of description, FIGS. 1A-1C will be generally described together. It is to be appreciated that cochlear implant system 100 may include other components that, for ease of illustration, have been omitted from FIGS. 1A-1C.

The cochlear implant system 100 comprises an external component 102 and an internal/implantable component 104, which is sometimes referred to as cochlear implant 104. In the example of FIGS. 1A-1C, the external component 102 comprises a behind-the-ear (BTE) sound processing unit 106 and a separate in-the-ear (ITE) unit 108. The BTE sound processing unit 106 is configured to be attached to, and worn adjacent to, the recipient's pinna 103, while the ITE unit 108 is configured to be worn in the ear canal 105 of the recipient. The pinna 103, the ear canal 105, and the eardrum generally form the outer ear 107 of the recipient. The recipient's hearing anatomy also includes the middle ear 109 (comprising the middle ear cavity 111 and the ossicular chain 113) and the cochlea 147.

The BTE sound processing unit 106 comprises one or more input elements/devices 113 for receiving input signals, such as sound signals. In this example, the one or more one or more input devices 113 include sound input devices 114 (e.g., microphones, telecoils, etc.) configured to capture/receive input signals, one or more auxiliary input devices 115 (e.g., audio ports, such as a Direct Audio Input (DAI), data ports, such as a Universal Serial Bus (USB) ports, cable ports, etc.), and a short-range wireless interface 116 (e.g., Bluetooth or BLE interface), each located in, on, or near a housing 117 of the sound processing unit 106.

The sound processing unit 106 also includes, for example, at least one battery 118, radio-frequency (RF) interface circuitry (transceiver) 119, and a processing module 120. The processing module 120 comprises at least one processor 121 and at least one memory device (memory) 122. Memory 122 includes sound processing logic 123. The sound processing logic 123, when executed by the at least one processor 121, causes the at least one processor 121 to perform sound processing operations (e.g., convert sound signals into stimulation control signals).

Memory 122 may comprise any suitable volatile or non-volatile computer readable storage media including, for example: Non-Volatile Memory (NVM), Ferroelectric Random Access Memory (FRAM), random access memory (RAM), cache memory, persistent storage (e.g., semiconductor storage device, read-only memory (ROM), erasable programmable read-only memory (EPROM), flash memory, etc.), or any other computer readable storage media that is capable of storing program instructions or digital information. The processing module 120 may be implemented, for example, on one or more printed circuit boards (PCBs).

It is to be appreciated that the arrangement for processing module 120 in FIG. 1B is merely illustrative and that the techniques presented herein may be implemented with a number of different processing arrangements. For example, the processing module 120 may be implemented by any of, or a combination of, one or more processors (e.g., one or more Digital Signal Processors (DSPs), one or more uC cores, etc.), firmware, software, digital logic gates in one or more application-specific integrated circuits (ASICs), etc.

Returning to the example embodiment of FIGS. 1A-1C, the external component 102 also comprises the ITE component 108 configured to removably inserted in the ear canal 105. That is, when in use, the ITE component 108 is inserted into the ear canal 105 of the recipient. When not in use (e.g., when the recipient 101 is sleeping), the ITE component 108 is removed from the ear canal 105

The ITE component 108, which is shown in greater detail in FIG. 1C, comprises a body 124 arranged to be inserted into the ear canal. As described further below, the body 124 is shaped to conform to the anatomical shape of the ear canal 105. Also as described below, in certain embodiments, the body 124 comprises an manufactured ear mold. For ease of reference, FIGS. 1A-1C will be described with reference to the use of an ear mold 124.

Disposed in, on, partially-in, partially-on (e.g., attached to) the body 124 is an in-the-ear (ITE) coil 126. As shown in FIG. 1C, the ear mold 124 includes a receptacle (cutout) 128 in which the ITE coil 126 is positioned and a vent 130 extending through the elongate length of the ear mold 124. The ITE coil 126 is connected to the sound processing unit 106, namely the RF transceiver 119, via a cable/wire 132 and circuitry (e.g., wiring) within the ear mold 124. It is to be appreciated that vent 130 is merely illustrative and that ear molds can be formed without a vent. It is also to be appreciated that an ITE component in accordance with embodiments presented herein may include other components beyond those shown in FIG. 1C. For example, the ITE component could also include, for example, an acoustic receiver or other acoustic outlet, microphone(s) or other sound input devices, a processing module, or other components typically integrated in a BTE sound processing unit and/or an off-the-ear (OTE) sound processing unit. For example, in alternative embodiments, the BTE sound processing unit 106 and the ITE component 108 could be integrated into a single component configured to be at least partially inserted into the ear canal of a recipient.

The implantable component (cochlear implant) 104 comprises an implant body (main module) 134, a lead region 136, and an intra-cochlear stimulating assembly 138, all configured to be implanted under the skin/tissue (tissue) of the recipient. The implant body 134 generally comprises a hermetically-sealed housing 140 in which RF interface circuitry 142 and a stimulator unit 144 are disposed. The implant body 134 also includes an internal/implantable coil 146 that is generally external to the housing 134, but which is connected to the RF interface circuitry 142 via a hermetic feedthrough (not shown in FIG. 1B).

As shown in FIG. 1A, the implant body 134 is configured to be implanted fully/completely within a cavity 135 formed in the mastoid bone 137 of the recipient 101. The cavity 135 is formed adjacent to the ear canal 105 of the recipient such that, as shown in FIG. 1A, the implant body 134 is adjacent to a superior surface/wall of the ear canal 105. Once implanted, the implant body 134, and thus the implantable coil 126, are separated from the ear canal by tissue 139 (e.g., the tissue forming the superior wall of the ear canal 105).

As noted, in the example of FIG. 1A, the implant body 134 and implantable coil 126 are shown adjacent to a superior wall of the ear canal. It is to be appreciated that this specific location of the implant body 134 and implantable coil 126 is merely illustrative and that the implant body 134 and/or the implantable coil 126 could be implanted at different locations within the recipient. For example, in an alternative embodiment, the implant body 134 and/or the implantable coil 126 could be positioned in the middle ear cavity 111 (e.g., adjacent a distal end of the ear canal 105 formed by the eardrum).

Returning to the example of FIGS. 1A-1C, the stimulating assembly 138 is configured to be at least partially implanted in the recipient's cochlea 147. Stimulating assembly 138 includes a plurality of longitudinally spaced intra-cochlear electrical stimulating contacts (electrodes) 148 that collectively form a contact or electrode array 150 for delivery of electrical stimulation signals (current signals) to the recipient's auditory system. Stimulating assembly 138 extends through an opening in the recipient's cochlea 147 (e.g., a cochleostomy, the round window, etc.) and has a proximal end connected to stimulator unit 144 via lead region 136 and a hermetic feedthrough (not shown in FIG. 1B). Lead region 136 includes a plurality of conductors (wires) that electrically couple the electrodes 148 to the stimulator unit 144.

As noted, the cochlear implant system 100 includes the ITE coil 126 and the implantable coil 146. The coils 126 and 146 are typically wire antenna coils each comprised of multiple turns of wire (e.g., single-strand or multi-strand platinum or gold wire). When the coils 126 and 146 are operationally aligned with one another, the coils are inductively coupled together in a manner that forms a transcutaneous closely-coupled wireless link between the coils. The closely-coupled wireless link can be used to, for example, transfer power and/or data through tissue 139. In the example of FIGS. 1A-1B, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIGS. 1A-1C illustrate only one example arrangement.

As noted above, sound processing unit 106 includes the processing module 120. The processing module 120 is configured to convert input sound signals (e.g., sound signals captured by microphones 114) into stimulation control signals for use in stimulating a first ear of a recipient (i.e., the processing module 120 is configured to perform sound processing on sound signals received at the sound processing unit 106). Stated differently, the processing module 120 (e.g., one or more processing elements implementing firmware, software, etc.) is configured to convert received sound signals into stimulation control signals that represent electrical stimulation for delivery to the recipient. The sound signals that are processed and converted into stimulation control signals may be audio signals received via the sound input devices 114, signals received via the auxiliary input devices 115, signals received via the short-range wireless interface 116, etc.

In the embodiment of FIG. 1B, the stimulation control signals are provided to the RF interface circuitry 119, which transcutaneously transfers the stimulation control signals (e.g., in an encoded manner) to the implantable component 104 via ITE coil 126 and implantable coil 146. That is, the stimulation control signals are received at the RF interface circuitry 142 (in implant body 134) via implantable coil 146 and provided to the stimulator unit 144. The stimulator unit 144 is configured to utilize the stimulation control signals to generate electrical stimulation signals (e.g., current signals) for delivery to the recipient's cochlea 147 via one or more of the stimulating contacts 1486. In this way, cochlear implant system 100 electrically stimulates the recipient's auditory nerve cells, bypassing absent or defective hair cells that normally transduce acoustic vibrations into neural activity, in a manner that causes the recipient to perceive one or more components of the sound signals received at the sound processing unit 106.

As noted, when the cochlear implant system 100 is in operation, the ITE coil 126 and the implantable coil 146 should be operationally aligned with one another to enable the transcutaneous transfer of power and data between ITE component 108 and implantable component 104. More specifically, in operation, the RF interface circuitry 119 is configured to drive the ITE coil 126 with current (via cable 132) that causes the ITE coil 126 to generate/emit a magnetic field that is generally represented in FIG. 1C by flux lines 152 (e.g., the emitted magnetic field can be visualized as flux lines that emanate from a center of the ITE coil 126). When the emitted magnetic field passes through the implantable coil, a current is induced in the implantable coil 146 that, in turn, can be used as data for stimulation of the recipient and/or for power (e.g., for operational power, to charge an implantable battery, etc.).

The amount of current induced in the implantable coil 146 is related to the total magnetic flux enclosed by the area of the implantable coil 146 at a given time (i.e., the total magnetic flux linking a winding is proportional to the current through that winding). The total magnetic flux enclosed by the area of the implantable coil 146 largely depends on the relative positioning, in terms of location (proximity) and orientation, of the ITE coil 126 and the implantable coil 146. That is, in general, the total magnetic flux enclosed by the area of the implantable coil 146 will increase the more closely the ITE coil 126 and the implantable coil 146 are physically to one another, and the more parallel the ITE coil 126 and the implantable coil 146 are to one another. Therefore, for efficient transcutaneous transmission of power and/or data, it is generally desirable that the ITE component 108 be positioned into the ear canal 105 so that the ITE coil 126 and the implantable coil 146 have a specific relative positioning (e.g., a specific physical proximity and orientation relative to one another). More generally stated, the ITE coil 126 and the implantable coil 146 should have selected inductive coupling with one another (mutual inductance), such as an inductive coupling strength that is greater than a minimum threshold level.

It is to be appreciated that the ITE component 108 would be worn in the ear canal 105 for only certain periods of time (e.g., during the day) and would be removed when not in use (e.g., when the recipient is sleeping). As such, the recipient or other user will have to insert the ITE component 108 into the ear canal 105 one or more times each day. As noted, obtaining the specific relative positioning of ITE coil 126 and the implantable coil 146, particularly in a repeatable manner (e.g., each time the ITE component 108 is inserted) is important to the efficient operation of cochlear implant system 100, but is also difficult in conventional arrangements.

As such, presented herein are techniques that for identifying an optimal placement of the ITE coil 126 in the ear canal 105 and for ensuring that the ITE coil 126 can to be inserted into the ear canal 105 such that the ITE coil is repeatedly/consistently appropriately positioned relative to implantable coil 146. In certain embodiments, the techniques presented herein enable the manufacture of an ear mold, such as ear mold 124, that is configured to have the ITE coil 126 removably positioned therein. The ear mold 124 is manufactured so that, when the ear mold 124 is inserted into the ear canal 105, the ITE coil 126 and the implantable coil 146 will have a specific/selected (e.g., optimal) relative positioning. As described further below, the optimal placement of the ITE coil 126 within the ear canal 105 is determined based on radio frequency (RF) measurements and the anatomy of the recipient of the cochlear implant system 100.

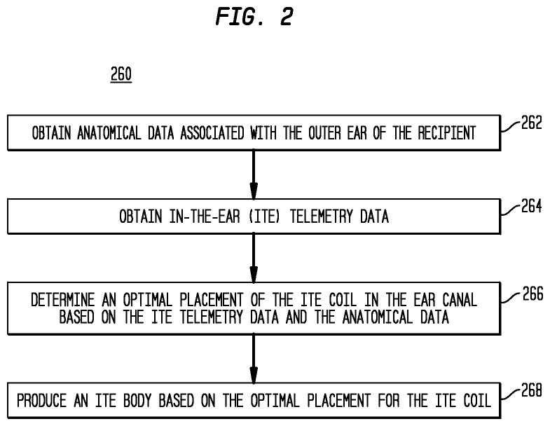

FIG. 2 is a flowchart illustrating an example method 260, in accordance with certain embodiments presented herein. For ease of description, the example method 260 will be described with reference to the cochlear implant system 100 of FIGS. 1A-1C implanted in recipient 101.

Method 260 begins at 262 where a computing device obtains anatomical data associated with the outer ear 107 (e.g., at least the ear canal 105) of the recipient 101. In accordance with embodiments presented herein, the anatomical data associated with the outer ear 107 of the recipient 101, which is sometimes referred to herein as “recipient anatomical data,” can be obtained and/or generated in any of a number of different manners.

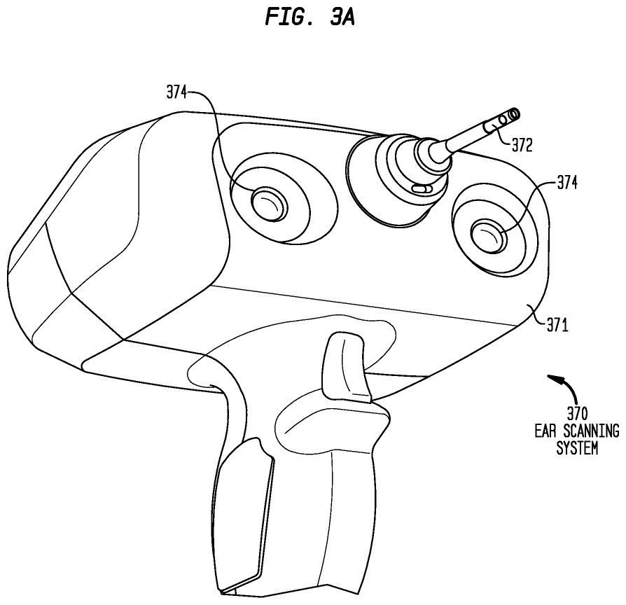

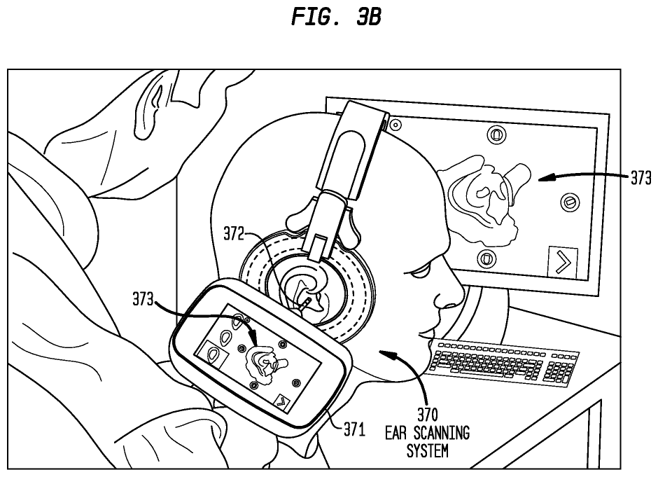

In one example, a three-dimensional (3D) imaging scan of the empty ear canal 105 is performed to obtain the recipient anatomical data. In such examples, a 3D ear scanning system is used to record the shapes of, for example, the pinna 103 and the ear canal 105. FIGS. 3A and 3B generally illustrates an example ear scanning system 370 configured to perform a 3D imaging scan of the outer ear 107. FIG. 3A is a perspective view of the ear scanning system 370 only, while FIG. 3B is perspective view of the ear scanning system 370 in use to scan the outer ear 107 of the recipient 101.

As shown in FIGS. 3A and 3B, the scanning system 370 comprises scanner body 371 and a probe 372. The scanner body 371 includes, among other components, cameras 374 configured to capture images of the outer ear 107. The probe 372 extends from the scanner body 371 and is configured to be inserted into the ear canal 105. The probe 372 also includes one or more optical elements (e.g., lasers, cameras, or other imaging component) that are configured to capture 3D images of the ear canal 105 (e.g., when the probe 372 is inserted therein) and the pinna 103. The 3D images can be analyzed to generate 3D anatomical data (e.g., 3D point cloud) of the ear canal 105 and the pinna 103. The 3D anatomical data can be used to, for example, generate a 3D representation of the outer ear 107, which is sometimes referred to herein as a 3D virtual model of the outer ear. Two example 3D representations of the outer ear 107 (e.g., two virtual models), which are generated from the 3D anatomical data, are shown in FIG. 3B as 3D representations 373.

In certain embodiments, the 3D image scanning could be performed after implantation of the implantable component 104 in the recipient 101. In other embodiments, the 3D ear scanning could also or alternatively be performed preoperatively and the 3D anatomical data could be analyzed to determine whether the cochlear implant system arrangement shown in FIGS. 1A-1C (or another implant arrangement) would be feasible for the recipient 101 (e.g., feasible from the ear canal dimensions perspective).

It is to be appreciated that the use of 3D image scanning to obtain the anatomical data associated with the outer ear 107 is merely illustrative and that the recipient anatomical data can be obtained in other manners. For example, the recipient anatomical data can also or alternatively be obtained via medical imaging, such as X-rays, ultrasounds, computed tomography (CT) scans, magnetic resonance imaging (MRI), echography, nuclear medicine imaging, including positron-emission tomography (PET), etc. In certain examples, a combination of different techniques (e.g., 3D image scanning and CT scanning) may be used to obtain the anatomical data associated with the outer ear 107 of the recipient.

Returning to the example of FIG. 2, at 264, a computing device obtains radio frequency (RF) data, sometimes referred to herein as telemetry data, associated with different potential “placements” or “positions” for the ITE coil 126 in the ear canal 105. As noted above, a “placement” or “position” for the ITE coil 126 includes both the location of the ITE coil 126 within the ear canal 105 (e.g., lateral/medial location, superior/inferior location, or anterior/posterior location of the ITE coil in the ear canal) and the orientation of the ITE coil 126 (e.g., angular position of the coil relative to sagittal, coronal, and or transverse planes).

The RF data associated with different potential placements for the ITE coil 126 is obtained by performing an in-ear RF scan relative to the implantable coil 146. As used herein, an RF scan is performed by inserting a test RF coil assembly (test coil assembly) into the ear canal 105 and measuring RF data (capturing RF measurements) indicating the strength of the inductive coupling between the test coil assembly and the implantable coil 146 at different relative positions there between (e.g., different orientations and physical proximities between the test coil assembly and the implantable coil). For example, the test coil assembly can be moved to different placements (locations and orientations) within the ear canal 105 and the strength of the inductive coupling between the test coil assembly and the implantable coil 146 can be determined/measured at each of the different placements. That is, an audiologist, health care professional, or other user inserts a test coil assembly 475 into the ear canal 105 and the system begins to capture RF data indicating the strength of the inductive coupling between the test coil assembly and the implantable coil 146 at different potential placements. The RF scan may be performed with or without hearing sensation present (e.g., while stimulating the recipient or without stimulating the recipient).

As noted, the coil inserted into the ear canal 105 to perform the RF scan is referred to as the test coil assembly 475. It is to be appreciated that this nomenclature is merely for ease of reference and that the test coil assembly 475 may be the same as the ITE coil 126 (e.g., the test coil assembly 475 may be the same as the ITE coil 126 can be the same coil, same type of coil, interchangeable coils, etc.).

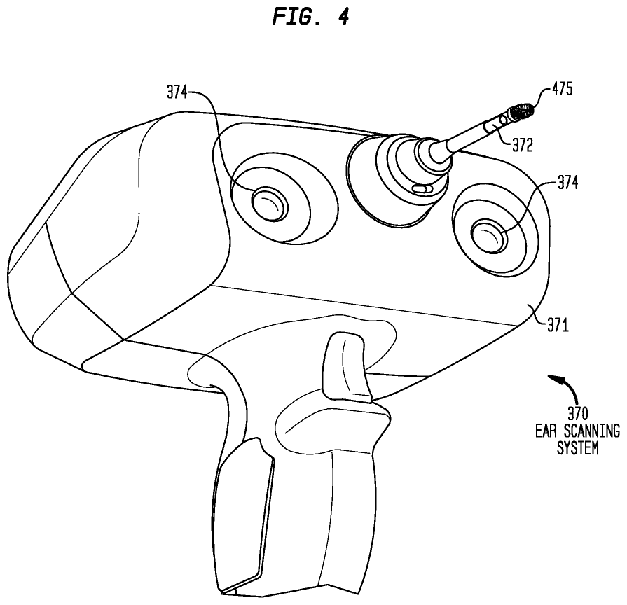

In one example shown in FIG. 4, a test coil assembly 475 can be attached to the distal end of the probe 372 of the ear scanning system 370 of FIGS. 3A and 3B. In such examples, test coil assembly 475 can be structurally and/or functionally the same as the ITE coil 126 that will be worn by the recipient and can be connected (e.g., via a wired or wireless connection) to the sound processing unit 106 (or an equivalent unit), which in connects (e.g., via a wired or wireless connection) to a computing device. In the specific example of FIG. 4, the mounting of the test coil assembly 475 at the distal end of the probe 372 may obstruct the optical elements of the scanner, but such obstruction is irrelevant since the optical elements are not, in this step, being used to determine the ear canal dimensions (e.g., the ear canal dimensions are known from the previous steps). However, in alternative embodiments, the test coil assembly 475 could be integrated into the probe 372 and/or attached to the probe in a manner such that the test coil assembly 475 does not obstruct the optical elements. Therefore, in certain such embodiments, the anatomical data associated with the outer ear 107 and the RF data associated with different potential placements for the ITE coil 126 could be obtained simultaneously or as part of the same insertion of the probe 372 into the ear canal 105.

In general, a mechanically repeatable jig is used to attach the test coil assembly to the ear scanning system 370 (or other device). In addition, the relative position of the test coil assembly to the scanning image is captured and made available to the modelling software.

As noted, once the test coil assembly 475 is inserted into the ear canal 105, the test coil assembly is moved to different placements and the system measures the strength of the inductive coupling between the test coil assembly 475 and the implantable coil 146 at the various different placements. Movement of the test coil assembly 475 to different locations and/or orientations could be facilitated in any of a number of different manners. For example, in one arrangement, the test coil assembly 475 could be manually re-positioned in the ear canal 105 (e.g., by manual moving the ear scanning system 370 such that the test coil assembly 475 is a different locations). In addition, the test coil assembly 475 could be mounted to the probe 371 via an articulable (rotatable) or pliable mount that allows the test coil assembly 475 to be manually turned to different orientations. In these embodiments, the position of the test coil assembly 475 in the ear canal 105 is captured and used in real time for reference/result.

In other embodiments, movement of the test coil assembly 475 to different locations and/or orientations could be partially or fully automated. For example, the test coil assembly 475 could be mounted to the probe 371 via a mechanical joint that allows the test coil assembly 475 to be automatically moved and/or rotated (e.g., via one or more step motors). In such embodiments, the system could execute a program to generate, for example, a complete 3D mapping (e.g., 3D heat map) of the magnetic field within the ear canal.

As noted, FIG. 4 illustrates an example in which the test coil assembly 475 is mounted to the probe 371. It is to be appreciated that this arrangement is merely illustrative and that the test coil assembly 475 could be inserted into the ear canal 105 without use of the probe 371.

For example, the test coil 475 could be connected to a passive pin/holder that is configured for manual, partially-automated, or fully-automated movement of the test coil assembly 475 to different placements within the ear canal 105.

In addition, during the RF scan (e.g., while capturing the RF data indicating the strength of the inductive coupling between the test coil assembly 475 and the implantable coil 146), the placement (e.g., location and orientation) of the test coil assembly 475 within the ear canal 105 is continually monitored. For example, in the embodiment of FIG. 4, the cameras 374 of the ear scanning system 370 are active and are configured to capture the position of the test coil assembly 475 inside the ear canal 105. The RF data indicating the strength of the inductive coupling between the test coil assembly 475 and the implantable coil 146 can be correlated with the real-time placement (e.g., location and orientation) of the test coil assembly 475 within the ear canal 105. Such correlation of the RF data and the real-time placement (e.g., location and orientation) of the test coil assembly 475 provides an indication of the inductive coupling strength as a function of the placement of the test coil assembly 475. Collectively, the RF data indicating the strength of the inductive coupling between the test coil assembly 475 and the implantable coil 146 and the real-time placement (e.g., location and orientation) of the test coil assembly 475 within the ear canal 105, when correlated with one another, is referred to herein as “ITE telemetry data” or simply “telemetry data.”

In certain embodiments, the ITE telemetry data could be provided, in real-time, to a user (e.g., using audible and/or visible indications) during the RF scan. In such embodiments, the ITE telemetry data, along with the anatomy of the recipient (e.g., anatomical, comfort, retention, etc. considerations), is used by the user to make a determination as to the optimal placement for the ITE coil 126 (e.g., manually select a position for the ITE coil based on the real-time RF data and the recipient's anatomical data). As noted, the optimal placement for the ITE coil 126 in the ear canal 105 is a placement in which the measured inductive coupling strength between the test coil assembly 475 and the implantable coil 146 was greater than a minimum threshold level and a placement that is anatomically appropriate for the recipient (e.g., accounts for the anatomical characteristics of the ear canal, including anatomical characteristics affecting retention, comfort, or other recipient-specific considerations).

In one such example, the real-time position of the test coil assembly 475 is recorded and displayed, at a display screen, along with a virtual model of the outer ear of the recipient during the RF scan (e.g., the real-time position of the test coil assembly 475 is displayed within a 3D model of the recipient's outer ear, which is generated from the recipient's anatomical data). While the RF scan is performed, the ITE telemetry data is also displayed in real-time, to the user. As such, the user can visualize the position of test coil assembly 475 with the ear canal 105, in real-time, as well as the strength of the inductive coupling. Using these displays, the user can determine the positioning of the test coil assembly 475 that provides a sufficient inductive coupling with the implantable coil 146, but which is also suitable for the specific anatomy of the recipient's ear canal 105 (e.g., balance inductive coupling strength with anatomical, comfort, retention, or other recipient-specific considerations). Once the optimal placement for the ITE coil 126 is determined, this position can be recorded (e.g., the user presses a button/trigger).

The above illustrates a real-time use of the ITE telemetry data to determine the optimal placement for the ITE coil 126. In alternative embodiments, this process may be fully or partially automated. For example, a full RF scan can be completed and the resulting ITE telemetry data is provided to a computing device. The computing device can be configured to analyze the ITE telemetry data and correlate the ITE telemetry data with the recipient's anatomical data. The computing device can then determine the optimal placement for the ITE coil 126 and/or provide several recommended optimal placements for the ITE coil 126. In certain examples, the determined the optimal placement, or recommended optimal placements, could be displayed at a display screen for evaluation by a user (e.g. determine whether the optimal placement, or recommended optimal placements are suitable for the specific anatomy of the recipient's ear canal).

In one such example, the ITE telemetry data could be logged at a computing device and analyzed for display as part of a 3D model of the recipient's outer ear. The computing device could provide recommendations for one or more placements of the ITE coil based on the ITE telemetry data and the anatomical data associated with the outer ear 107 of the recipient. A selected location is then determined (e.g., by the computing device in a fully automated process or based on additional inputs from a user in a partially automated process).

Returning to the example of FIG. 2, once the optimal placement for the ITE coil 126 is determined, at 268 the ITE component 108 is produced/fabricated based on the optimal placement. More specifically, as noted above, the ITE component 108 comprises a body 124 arranged (e.g., shaped, dimensioned, etc.) to be inserted into the ear canal (e.g., shaped to conform to the anatomical shape of the ear canal 105) and is configured to have the ITE coil 126 positioned therein. The body 124 is produced, based on the optimal placement for the ITE coil 126 and the recipient's anatomical data, so that when the ITE mold is fittingly inserted into the ear canal 105, the ITE coil 126 will be positioned at the previously determined optimal placement.

In general, the body 124 is produced such that it can only be inserted into the ear canal 105 in predetermined manner and will have a predetermined position when the body 124 is fittingly inserted into the ear canal. In addition, once inserted, the body 124 is substantially fixed/retained in the predetermined position in the ear canal. Therefore, the body 124 is produced/fabricated in a manner such that the ITE coil 126 within the body will have the previously determined optimal placement while the body 124 is retained at the predetermined position in the ear canal

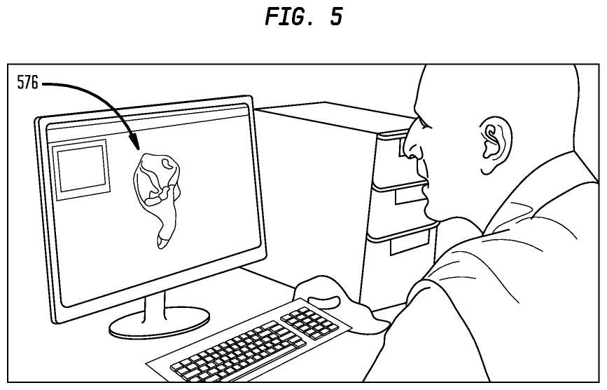

In certain embodiments, such as those shown in FIGS. 1A-1C, the body 124 comprises a manufactured ear mold. In one such embodiment, the recipient's anatomical data (e.g., 3D point cloud) and the optimal placement (optimal coil location and orientation) are provided to a mold manufacturing site (e.g., via electronic transfer). A 3D rendering software platform includes a 3D model of the ITE coil, including clearances and angle tolerances, along with data enabling suitable placement of coil in the ear mold with suitable venting. The end result is, as shown in FIG. 5, a 3D rendering 576 of an ear mold for the recipient, where the rendering includes a location for the coil with the ear mold.

The rendering, which is the link between the raw data and the manufacturing process, is used to produce/manufacture (e.g., via 3D printing) the ear mold into which the ITE coil 126 can be inserted. That is, in certain embodiments, the ear mold is manufactured so as to removably receive the ITE coil 126 in a manner that enables the ITE coil to be attached/detached from the ear mold. In this way, the ITE coil 126 and the ear mold can be separately serviced/replaced. However, in alternative embodiments, the ITE coil 126 can be integrated within the ear mold (e.g., cast or molded into the ear mold such that the coil not separable from the ear mold).

The ear mold, when completed, includes the ITE coil 126 with a location and angle that, when inserted into the ear canal 105, the ITE coil 126 will necessarily assume the previously determined optimal placement. In addition, the ear mold has an exterior/outside shape that repeatably positions the ITE coil 126 determined optimal placement each time the ITE component is inserted into the ear canal 105 (e.g., the ear mold can only be inserted into the ear canal 105, the ear mold will have a predetermined position when fittingly inserted into the ear canal, and the ear mold is substantially fixed/retained in the predetermined position in the ear canal).

In the general, the ITE telemetry data, the recipient's anatomical data, rendering, implant location, etc. can stored as part of the recipient's medical/hearing record for subsequent use, as needed. As such, future ITE components (e.g., ear molds with an ITE coil) can be produced quickly and accurately (repeatably) for recipient.

In summary, FIG. 2 illustrates an example method for manufacturing a recipient-specific ear mold configured to be inserted into an ear canal of a recipient. The ear mold includes, or is configured to receive, an ITE coil such that, when the ear mold is inserted into the ear canal, the ITE coil and an implantable coil with a selected relative positioning to one another. As such, the example of FIG. 2 facilitates an optimum RF link that provides high power efficiency transfer and extends battery life. The example of FIG. 2 also enables repeatable insertion by the recipient with consistent coil alignment, which enables the use of magnet-free implants (and coils). Since the ear mold confirms to the shape of the ear canal, the ear mold can avoids unpleasant sensations such as tickling, poor retention, occlusion, etc., while preserving residual hearing and providing ventilation to the ear canal.

As noted, the body 124 of the ITE component 108 can comprise a manufactured ear mold fabricated, for example, via 3D printing or other manufacturing process. However, it is also to be appreciated that the body 124 can be produced using other methods. For example, the body 124 could be generated using a direct ear impression material that is injectable into the ear canal 105 around the ITE coil 126 (at the previously determined optimal placement). Following the initial injection, such direct ear impression materials subsequently become sufficient structurally stable (e.g., harden) for use as the ITE component thereafter.

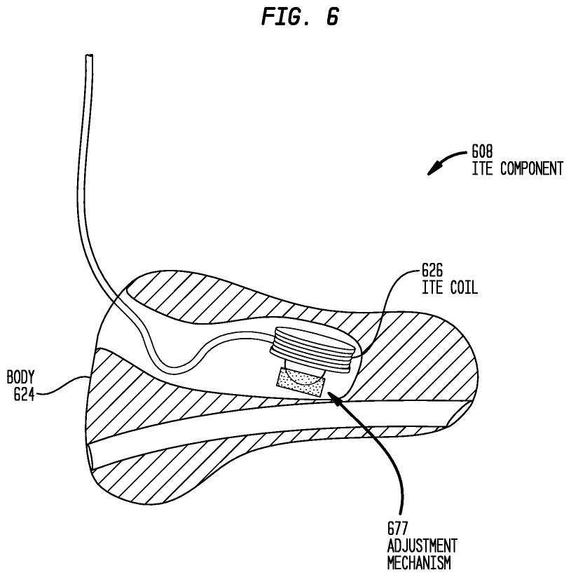

In the example of FIGS. 1A-1C, the ITE coil 126 can be directly attached to the body 124. However, in other embodiments, an ITE coil can be attached to the body via an adjustment mechanism, such as an articulable joint (e.g., ball joint), one or more adjustment screws, etc. For example, FIG. 6 illustrates an example ITE component 608 comprising a body 624 and an ITE coil 626. In this example, the ITE coil 626 is attached to the body via an adjustment mechanism 680. The use of the adjustment mechanism 677 to attach the ITE coil 626 to the body 624 enables adjustment to the location and/or orientation of the ITE coil 626, following attachment of the ITE coil to the body. These adjustments may be performed, for example, during fitting, testing, etc., and can be made based on RF measurements made when the example ITE component 608 is positioned in a recipient's ear canal.

In general, the body of an ITE component in accordance with embodiments is produced/formed such that, when inserted into the ear canal, the ITE coil within the ITE component will be self-aligned with (automatically aligned with), and sufficiently inductively coupled with, an implantable coil adjacent the ear canal. This self-alignment of the ITE coil with the implantable coil is facilitated by the arrangement (e.g., shape) of the body and the anatomical structures/features of the outer ear. That is, as noted above, the body of an ITE component in accordance with certain embodiments presented herein is produced such that it can only be inserted into the ear canal in a predetermined manner (due to the shape of the body and the anatomical features of the ear canal) and the body will have a predetermined position when the body is fittingly inserted into the ear canal. In addition, once inserted, the body is substantially fixed/retained in the predetermined position in the ear canal where the ITE coil is generally aligned with the implantable coil.

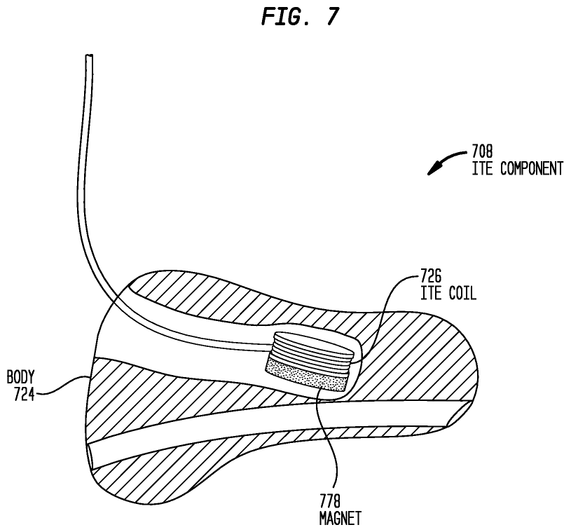

In certain embodiments, the self-alignment of the ITE coil can be supplemented with one or more auxiliary alignment features. In one example, the auxiliary alignment features may comprise a metallic or magnetic component (e.g., ferromagnetic metal, a permanent magnet, etc.) disposed in the ITE component that is configured to be magnetically attached to the implantable coil itself and/or a metallic or magnetic component implanted with the implantable coil. FIG. 7 illustrates an example ITE component 708 comprising a body 724, an ITE coil 726, and a magnet 778. In use, an arrangement such as shown in FIG. 7 would further ensure that the ITE coil 726 is consistently/repeatably aligned with the implantable coil each time the ITE component 708 is inserted into the ear canal.

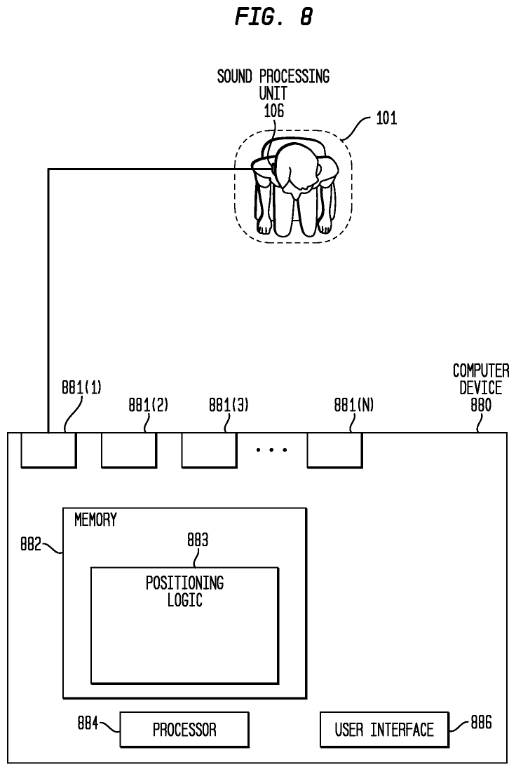

As noted above, aspects of the techniques presented herein may be performed by a computing device (e.g., desktop computer, laptop computer, tablet computer, mobile phone, fitting system, etc.). FIG. 8 illustrates one example computing device 880 that can be used to determine the optimal position of an ITE coil, in accordance with certain embodiments presented herein. As shown, computing device 880 comprises a plurality of interfaces/ports 881(1)-881(N), a memory device (memory) 882, at least one processor 884, and a user interface 886.

The interfaces 881(1)-881(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 8, interface 881(1) is connected to a test coil assembly, such as test coil assembly 475, via a sound processing unit (or equivalent device) via a wired or wireless connection (e.g., telemetry, Bluetooth, etc.).

The user interface 886 includes one or more output devices, such as a liquid crystal display (LCD) and a speaker, for presentation of visual or audible information to a clinician, audiologist, or other user. The user interface 886 may also comprise one or more input devices that include, for example, a keypad, keyboard, mouse, touchscreen, etc.

The memory 882 comprises positioning logic 883. The memory 882 may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices.

The at least one processor 884 is, for example, a microprocessor or microcontroller that executes instructions for the positioning logic 883. Thus, in general, the memory 880 may comprise one or more tangible (non-transitory) computer readable storage media (e.g., a memory device) encoded with software comprising computer executable instructions and when the software is executed (by the processor 884), it is operable to perform the techniques described herein. For example, the positioning logic 883 may be executed to: display recipient anatomical data and/or one or more displays generated from the recipient anatomical data (e.g., a virtual model of the outer ear), display ITE telemetry data and/or one or more displays generated from the ITE telemetry data, determine the optimal placement of an ITE coil in the ear canal, provide suggested placements for an ITE coil in the ear canal, etc.

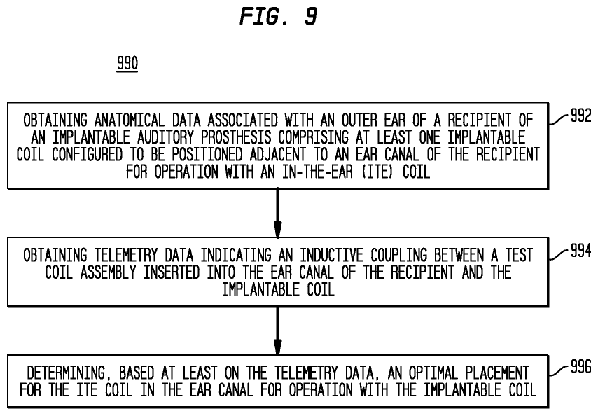

FIG. 9 is a flowchart of a method 990 in accordance with certain embodiments presented herein. Method 990 begins at 992 where anatomical data associated with an outer ear of a recipient of an implantable auditory prosthesis is obtained. The implantable auditory prosthesis comprises at least one implantable coil configured to be positioned adjacent to an ear canal of the recipient for operation with an in-the-ear (ITE) coil. At 994, telemetry data indicating an inductive coupling between a test coil assembly inserted into the ear canal of the recipient and the implantable coil is obtained. At 996, based at least on the telemetry data, an optimal placement for the ITE coil in the ear canal for operation with the implantable coil is determined.

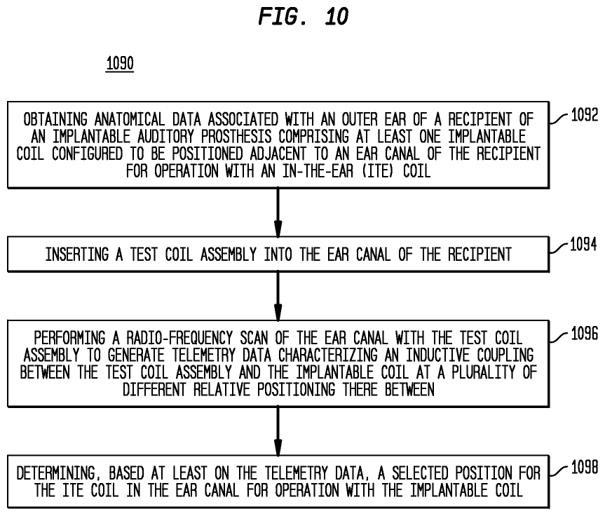

FIG. 10 is a flowchart of a method 1090 in accordance with certain embodiments presented herein. Method 1090 begins at 1092 where anatomical data associated with an outer ear of a recipient of an implantable auditory prosthesis is obtained. The implantable auditory prosthesis comprises at least one implantable coil configured to be positioned adjacent to an ear canal of the recipient for operation with an in-the-ear (ITE) coil. At 1094, a test coil assembly is inserted into the ear canal of the recipient. At 1096, a radio-frequency scan of the ear canal is performed with the test coil assembly to generate telemetry data characterizing an inductive coupling between the test coil assembly and the implantable coil at a plurality of different relative positioning there between. At 1098, based at least on the telemetry data, a selected position for the ITE coil in the ear canal for operation with the implantable coil is determined.

Embodiments of the techniques presented herein have been primarily described above with reference to an ITE component of an example cochlear implant system However, as noted elsewhere herein, it is to be appreciated the embodiments presented herein may be used to determine the optimal placement of an ITE coil for other types of systems, including other auditory prostheses, such as other cochlear implant system arrangements, bone conduction devices, direct acoustic stimulators, electro-acoustic prostheses, auditory brain stimulators, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

It is to be appreciated that the embodiments presented herein are not mutually exclusive and that the various embodiments may be combined with another in any of a number of different manners.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

1. A method, comprising:
obtaining anatomical data associated with an outer ear of a recipient of an implantable auditory prosthesis comprising at least one implantable coil configured to be positioned adjacent to an ear canal of the recipient for operation with an in-the-ear (ITE) coil;
obtaining telemetry data indicating an inductive coupling between a test coil assembly inserted into the ear canal of the recipient and the at least one implantable coil; and
determining, based at least on the telemetry data, an optimal placement for the ITE coil in the ear canal for operation with the at least one implantable coil.

2. The method of claim 1, wherein obtaining the anatomical data associated with the outer ear of the recipient comprises:
performing a three-dimensional (3D) imaging scan of the outer ear of the recipient.

3. The method of claim 1, wherein obtaining the anatomical data associated with the outer ear of the recipient comprises:
obtaining medical imaging of the outer ear of the recipient.

4. The method of claim 1, wherein obtaining telemetry data indicating the inductive coupling between the test coil assembly inserted into the ear canal of the recipient and the at least one implantable coil comprises:
inserting the test coil assembly into the ear canal of the recipient; and
performing a radio-frequency scan of the ear canal with the test coil assembly.

5. The method of claim 4, wherein performing the radio-frequency scan of the ear canal comprises:
positioning the test coil assembly at different positions within the ear canal; and
obtaining radio-frequency measurements for each of a plurality of the different positions of the test coil assembly within the ear canal.

6. The method of claim 5, wherein positioning the test coil assembly at different positions within the ear canal comprises:
positioning the test coil assembly at a number of different locations within the ear canal or at a number of different orientations relative to the at least one implantable coil.

7. The method of claim 6, wherein positioning the test coil assembly at different positions within the ear canal comprises:
positioning the test coil assembly at a number of different locations within the ear canal and at a number of different orientations relative to the at least one implantable coil.

8. The method of claim 5, wherein positioning the test coil assembly at different positions within the ear canal comprises:
manually positioning the test coil assembly at a number of different locations within the ear canal or at a number of different orientations relative to the at least one implantable coil.

9. The method of claim 5, wherein positioning the test coil assembly at different positions within the ear canal comprises:
automatically positioning the test coil assembly at a number of different locations within the ear canal or at a number of different orientations relative to the at least one implantable coil.

10. The method of claim 1, wherein determining the optimal placement for the ITE coil in the ear canal for operation with the implantable coil comprises:
determining a selected location of the ITE coil in the ear canal and a selected orientation of the ITE coil relative to the at least one implantable coil.

11. The method of claim 1, wherein determining the optimal placement for the ITE coil in the ear canal for operation with the at least one implantable coil includes:
generating at least one of a real-time audible or real-time visible representation of the telemetry data;
generating a real-time visible representation of a position of the test coil assembly within the ear canal; and
determining the optimal placement based on the at least one of the real-time audible or real-time visible representation of the telemetry data and the real-time visible representation of a position of the test coil assembly within the ear canal.

12. The method of claim 11, wherein generating a real-time visible representation of the position of the test coil assembly within the ear canal comprises:
generating, based on the anatomical data, a virtual model of an outer ear of the recipient including the ear canal;
displaying the virtual model of the outer ear of the recipient at a display screen;
monitoring the position of the test coil assembly in the ear canal; and
displaying, within the virtual model, the real-time visible representation of the position of the test coil assembly.

13. The method of claim 1, wherein determining the optimal placement for the ITE coil in the ear canal for operation with the at least one implantable coil comprises:
determining the optimal placement for the ITE coil based on the telemetry data and the anatomical data.

14. The method of claim 13, wherein determining the optimal placement for the ITE coil based on the telemetry data and the anatomical data comprises:
generating, at a computing device, one or more suggested positions for the ITE coil within the ear canal; and
generating a visible indication of the one or more suggested positions for the ITE coil within the ear canal.

15. The method of claim 1, further comprising:
forming an ITE component comprising a body and ITE coil for insertion into the ear canal, wherein the ITE component has an arrangement such that, when the body is inserted into the ear canal, the ITE coil will be situated at the optimal placement.

16. The method of claim 15, wherein the body comprises an ear mold, and wherein forming the ITE component comprises:
forming the ear mold based on the anatomical data and the telemetry data.

17. The method of claim 16, wherein forming the ear mold based on the anatomical data and the telemetry data comprises:
rendering, at a computing device, a three-dimensional (3D) model of the ear mold; and
manufacturing the ear mold from the 3D model of the ear mold rendered at the computing device.

18. The method of claim 16, wherein forming the ITE component comprises:
injecting a direct ear impression material into the ear canal around the ITE coil situated at the optimal placement.

19. An in-the-ear (ITE) component for use with an implantable coil configured to be positioned adjacent to an ear canal of a recipient, comprising:
a body arranged to be inserted in the ear canal of the recipient; and
an ITE coil attached to the body,
wherein the body has a shape such that, when the body is fittingly inserted into the ear canal, the ITE coil and the implantable coil have a predetermined relative positioning determined based on telemetry data obtained from within the ear canal of the recipient.

20. The ITE component of claim 19, wherein the ITE coil is removably attached to the body.

21. The ITE component of claim 19, wherein the ITE coil is integrated into the body.

22. The ITE component of claim 19, wherein the ITE coil is attached to the body via an adjustment mechanism configured to enable adjustments to a placement of the coil in the ear canal.

23. The ITE component of claim 19, wherein the body includes at least one of a metallic or magnetic component for magnetic coupling with a metallic or magnetic component implanted adjacent to the implantable coil.

24. The ITE component of claim 19, wherein the body comprises an ear mold generated based on the telemetry data and anatomical data associated with the ear canal of the recipient.

25. An auditory prosthesis system comprising the ITE component of claim 19, and an implantable component configured to be implanted in a recipient, wherein the implantable component includes the implantable coil.

26. The auditory prosthesis system of claim 25, wherein the auditory prosthesis system is a cochlear implant system comprising a stimulating assembly configured to be positioned in a cochlea of the recipient.

27-40. (canceled)
